(19) United States
(12) Patent Application Publication
(10) Pub. No.: US 2021/0162413 A1
(43) Pub. Date: Jun. 3, 2021

(54) MICROFLUIDIC IMMUNOASSAYS

(71) Applicant: HEWLETT-PACKARD DEVELOPMENT COMPANY, L.P., Spring, TX (US)

(72) Inventors: Alexander N. GOVYADINOV, Corvallis, OR (US); David P. MARKEL, Corvallis, OR (US); Erik D. TORNIAINEN, Corvallis, OR (US); Pavel KORNILOVICH, Corvallis, OR (US)

(73) Assignee: HEWLETT-PACKARD DEVELOPMENT COMPANY, L.P., Spring, TX (US)

(21) Appl. No.: 17/047,941

(22) PCT Filed: Jun. 18, 2018
(86) PCT No.: PCT/US2018/038136

§ 371 Date: Oct. 15, 2020

(51) Int. Cl. B01L 3/00; G01N 33/543

(52) U.S. Cl. CPC B01L 3/502753; B01L 3/50273; B01L 3/502761; G01N 33/54366; B01L 2400/0415; B01L 2300/0681; B01L 2300/0864; B01L 2300/1827; B01L 2200/0647

(57) ABSTRACT

A microfluidic immunoassay platform may include a substrate, a microfluidic channel in the substrate, a first set of functionalized structures along the channel, a second set of functionalized structures along the channel and an electrically driven fluid actuator contained on the substrate to move fluid containing at least one analyte along the channel through the first set of functionalized structures and through the second set of functionalized structures.

### BACKGROUND

Immunoassays are biochemical tests that detect and measure the presence or concentration of a macromolecule or a small molecule in a solution using an antibody or an antigen. The molecule or molecules detected by the immunoassay, the “analyte,” may comprise a protein or other kinds of molecules. Immunoassays are often utilized to identify and measure analytes in biological liquids, such as serum or urine, for medical or research purposes.

### BRIEF DESCRIPTION OF THE DRAWINGS

FIG. 1 is a top sectional view illustrating portions of an example microfluidic immunoassay platform.

FIG. 2 is a flow diagram of an example immunoassay method.

FIG. 3A is a top sectional view illustrating portions of an example microfluidic immunoassay platform.

FIG. 3B is a side sectional view of the platform of FIG. 3A.

FIG. 4A is a top sectional view illustrating portions of an example microfluidic immunoassay platform.

FIG. 4B is a side sectional view of the platform of FIG. 4A.

FIG. 5 is a top sectional view illustrating portions of an example microfluidic immunoassay platform.

FIG. 6 is a schematic diagram illustrating an example set of functionalized structures.

FIG. 7 is a flow diagram of an example method for forming an immunoassay platform and using the immunoassay platform.

FIG. 8A is a top sectional view illustrating portions of an immunoassay platform during deposition of a first set of functionalized structures as part of forming the immunoassay platform.

FIG. 8B is a top sectional view illustrating portions of the immunoassay platform of FIG. 8A during deposition of a second set of functionalized structures as part of forming the immunoassay platform.

FIG. 8C is a top sectional view illustrating portions of the immunoassay platform of FIG. 8B during movement of an analyte-containing fluid through the first set of functionalized structures and the second set of functionalized structures.

FIG. 9A is a top sectional view illustrating portions of an immunoassay platform during deposition of a first set of functionalized structures as part of forming the immunoassay platform.

FIG. 9B is a top sectional view illustrating portions of the immunoassay platform of FIG. 9A during deposition of a second set of functionalized structures as part of forming the immunoassay platform.

FIG. 9C is a top sectional view illustrating portions of the immunoassay platform of FIG. 9B during deposition of a third set of functionalized structures has part of forming the immunoassay platform.

FIG. 10A is a top sectional view illustrating portions of an immunoassay platform during deposition of a first set of functionalized structures as part of forming the immunoassay platform.

FIG. 10B is a top sectional view illustrating portions of the immunoassay platform of FIG. 10A during deposition of a second set of functionalized structures as part of forming the immunoassay platform.

FIG. 10C is a top sectional view illustrating portions of the immunoassay platform of FIG. 10B during deposition of a third set of functionalized structures has part of forming the immunoassay platform.

FIG. 11 is a top sectional view illustrating portions of an example microfluidic immunoassay platform.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION OF EXAMPLES

Disclosed herein are example microfluidic immunoassay platforms and methods that facilitate efficient and economical immunoassays. The disclosed immunoassay platforms and methods may provide high throughput with the ability to multiplex a greater number of analytes in a single test. The disclosed immunoassay platforms and methods provide such immunoassay multiplexing with a proportional lower degree of complexity and cost.

In contrast to immunoassays that are carried out utilizing magnetic functionalized beads, the disclosed immunoassay platforms and methods may offer greater flexibility in that the disclosed platforms and methods may be utilized with a larger variety of functionalized structures, such as non-magnetic beads and pillars. In contrast to immunoassays that are carried out with magnetic functionalized beads in a container or well plate, the disclosed immunoassay platforms and methods may be carried out on a platform having an electrically driven fluid actuator which controllably moves the solution containing the at least one analyte through and across the functionalized structures. In contrast to other immunoassays, the disclosed immunoassay platforms and methods may omit the step of sorting different assay beads after analyte binding when multiplexing different analytes.

The disclosed immunoassay platforms and methods utilize an electrically driven fluid actuator on a substrate to move a fluid containing at least one analyte along a channel through sets of functionalized structures. A “functionalized” structure is a structure that has been treated with a binding agent that binds to a specific molecule in a solution that may contain a complex mixture of molecules. In some implementations, the binding agent may be an antibody that binds to an epitope of an antigen analyte. In other implementations, the binding agent may be an antigen that binds to an antibody analyte. In some implementations, the binding agent, whether an antibody or an antigen, is chemically linkable to a detectable label. Such labels may emit radiation, produce a color change in a solution, fluoresce under light or, when induced, emit light. Such labels facilitate the detection of a bound analyte to measure the presence or concentration of the analyte in a solution, or other characteristics of the analyte.

The functionalized structures may have a variety of forms. In one implementation, the functionalized structure may comprise magnetic beads. In another implementation, the functionalized structures may comprise non-magnetic beads. In yet another implementation, the functionalized structures may comprise posts or pillars. The size and shape of the individual functionalized structures may be varied on a single platform to provide different immunoassay testing characteristics within or across the platform. The number, layout or arrangement, and density of the functionalized structures may be varied on a single platform to provide different immunoassay testing characteristics within or across the platform.

As will be appreciated, examples provided herein may be formed by performing various microfabrication and/or micromachining processes on a substrate to form and/or connect structures and/or components. Substrates forming the various fluidic components may comprise a silicon-based wafer or other such similar materials used for microfabricated devices (e.g., glass, gallium arsenide, quartz, sapphire, metal, plastics, etc.). Examples may comprise microfluidic channels, fluid actuators, and/or volumetric chambers. Microfluidic channels and/or chambers may be formed by performing etching, microfabrication processes (e.g., photolithography), or micromachining processes in a substrate. Accordingly, microfluidic channels and/or chambers may be defined by surfaces fabricated in the substrate of a microfluidic device. In some implementations, microfluidic channels and/or chambers may be formed by an overall package, wherein multiple connected package components combine to form or define the microfluidic channel and/or chamber.

In some examples described herein, at least one dimension of a microfluidic channel and/or capillary chamber may be of sufficiently small size (e.g., of nanometer sized scale, micrometer sized scale, millimeter sized scale, etc.) to facilitate pumping of small volumes of fluid (e.g., picoliter scale, nanoliter scale, microliter scale, milliliter scale, etc.). For example, some microfluidic channels may facilitate capillary pumping due to capillary force. In addition, examples may couple at least two microfluidic channels to a microfluidic output channel via a fluid junction.

The electrically driven fluid actuator used to drive or move the solution containing an analyte through and across the functionalized structures may enhance binding kinetics. In other words, the electrically driven fluid actuator may improve the ability of the analyte in the solution to come into contact with and bind to the binding agents on the surfaces of the functionalized structures. The electrically driven fluid actuator controls the rate at which the solution is moved through and across the functionalized structures. The electrically driven fluid actuator facilitates the provision of immunoassays on a single platform or chip, facilitating the provision of lab-on-chips.

The fluid actuator on the platform used to displace fluid through and across the functionalized structures, also on the platform, may comprise a thermal resistive fluid actuator, a piezo-membrane based actuator, and electrostatic membrane actuator, mechanical/impact driven membrane actuator, a magnetostrictive drive actuator, and electrochemical actuator, and external laser actuators (that form a bubble through boiling with a laser beam), other such microdevices, or any combination thereof.

In some implementations, electrically driven fluid actuator may comprise an inertial pump formed up on the platform that pumps fluid through and across the functionalized structures. In one implementation, the inertial pump may push fluid through and across the functionalized structures. In another implementation, the inertial pump may displace fluid so as to draw fluid through and across the functionalized structures.

As used herein, an inertial pump corresponds to a fluid actuator and related components disposed in an asymmetric position in a fluid channel, where an asymmetric position of the fluid actuator corresponds to the fluid actuator being positioned less distance from a first end of the fluid channel as compared to a distance to a second end of the fluid channel. Accordingly, in some examples, a fluid actuator of an inertial pump is not positioned at a mid-point of a fluid channel. The asymmetric positioning of the fluid actuator in the fluid channel facilitates an asymmetric response in fluid proximate the fluid actuator that results in fluid displacement when the fluid actuator is actuated. Repeated actuation of the fluid actuator causes a pulse-like flow of fluid through the fluid channel.

In some examples, an inertial pump includes a thermal actuator having a heating element (e.g., a thermal resistor) that may be heated to cause a bubble to form in a fluid proximate the heating element. In such examples, a surface of a heating element (having a surface area) may be proximate to a surface of a fluid channel in which the heating element is disposed such that fluid in the fluid channel may thermally interact with the heating element. In some examples, the heating element may comprise a thermal resistor with at least one passivation layer disposed on a heating surface such that fluid to be heated may contact a topmost surface of the at least one passivation layer. Formation and subsequent collapse of such bubble may generate flow of the fluid. As will be appreciated, asymmetries of the expansion-collapse cycle for a bubble may generate such flow for fluid pumping, where such pumping may be referred to as “inertial pumping.”

In other examples, the fluid actuator(s) forming an inertial pump or used to eject fluid through an ejection orifice or nozzle may comprise piezo-membrane based actuators, electrostatic membrane actuators, mechanical/impact driven membrane actuators, magnetostrictive drive actuators, electrochemical actuators, external laser actuators (that form a bubble through boiling with a laser beam), other such microdevices, or any combination thereof. In some implementations, the fluid actuators may displace fluid through movement of a membrane (such as a piezo-electric membrane) that generates compressive and tensile fluid displacements to thereby cause inertial fluid flow.

As will be appreciated, the fluid actuator forming the inertial pump may be connected to a controller, and electrical actuation of the fluid actuator by the controller may thereby control pumping of fluid. Actuation of the fluid actuator may be of relatively short duration. In some examples, the fluid actuator may be pulsed at a particular frequency for a particular duration. In some examples, actuation of the fluid actuator may be 1 microsecond (μs) or less. In some examples, actuation of the fluid actuator may be within a range of approximately 0.1 microsecond (μs) to approximately 10 milliseconds (ms). In some examples described herein, actuation of the fluid actuator includes electrical actuation. In such examples, a controller may be electrically connected to a fluid actuator such that an electrical signal may be transmitted by the controller to the fluid actuator to thereby actuate the fluid actuator. Each fluid actuator of an example microfluidic device may be actuated according to actuation characteristics. Examples of actuation characteristics include, for example, frequency of actuation, duration of actuation, number of pulses per actuation, intensity or amplitude of actuation, phase offset of actuation.

In other implementations, the electrically driven fluid actuator may be part of a fluid ejector that ejects droplets of fluid, creating a low pressure are sub-atmospheric pressure that draws fluid through and across the functionalized structures. In some implementations, the sub-atmospheric pressure may be ⅓ of an atmosphere. For example, in one implementation, fluid ejector may comprise a thermal resistor that vaporizes the adjacent fluid to create a bubble that displaces adjacent liquid to eject at least one drop of the liquid through an adjacent orifice, creating a low pressure that draws fluid through and across the functionalized structures.

Disclosed herein is an example microfluidic immunoassay platform that may include a substrate, a microfluidic channel in the substrate, a first set of functionalized structures along the channel, a second set of functionalized structures along the channel and an electrically driven fluid actuator contained on the substrate to move fluid containing at least one analyte along the channel through the first set of functionalized structures and through the second set of functionalized structures.

Disclosed herein is an example immunoassay method that includes providing a first set of functionalized structures and a second set of functionalized structures along a channel of a substrate and moving a fluid containing an analyte along the channel with an electrically driven fluid actuator contained on the substrate.

Disclosed herein is an example method for forming and using an immunoassay platform. The method comprises moving a first fluid containing a first set of functionalized beads along a channel in a substrate to deposit the first set of functionalized beads along the channel, moving a second fluid containing a second set of functionalized beads along the channel in the substrate to deposit the second set of functionalized beads along the channel and moving a third fluid containing at least one analyte through the first set of functionalized beads along the channel and through the second set of functionalized beads along the channel.

FIG. 1 schematically illustrates portions of an example microfluidic immunoassay platform 20. Platform 20 facilitates efficient and economical immunoassays. Platform 20 may provide high throughput with the ability to multiplex a greater number of analytes in a single test. Platform 20 may provide such immunoassay multiplexing with a proportional lower degree of complexity and cost.

In contrast to immunoassays that are carried out utilizing magnetic functionalized beads, platform 20 may offer greater flexibility in that platform 20 may be utilized with a larger variety of functionalized structures, such as non-magnetic beads and pillars. In contrast to immunoassays that are carried out with magnetic functionalized beads in a container or well plate, platform 20 may be carried out on a platform having an electrically driven fluid actuator which controllably moves the solution containing the at least one analyte through and across the functionalized structures. In contrast to other immunoassays, platform 20 may omit the step of sorting different assay beads after analyte binding when multiplexing different analytes. Platform 20 comprises substrate 22, microfluidic channel 24, sets 30-1 and 30-2 (collectively referred to as sets 30) of functionalized structures, and electrically driven fluid actuator 40.

Substrate 22 comprises at least one layer of material forming a foundation or base of platform 20. Substrate 22 may comprise a silicon-based wafer or die or a wafer or die from other such similar materials used for microfabricated devices (e.g., glass, gallium arsenide, plastics, etc.). In one implementation in which a detectable label is linked to an analyte bound to the functionalized structures or to the functionalized structures, at least portion 23 (shown by broken lines) of substrate 22 proximate or adjacent to the functionalized structures may be sufficiently translucent or transparent to facilitate optical detection of the detectable labels or their properties. For example, in some implementations, substrate 22 may completely surround channel 24, wherein portion 23 of the substrate 22 adjacent to channel 24 is transparent to facilitate optical sensing of florescence or luminescence of the detectable labels/markers/tags that become linked to the bound analyte or functionalized structures. In one such implementation, the portion 23 of substrate 22 that is transparent may be formed from a transparent glass material. In some implementations, the entirety of substrate 22 may be formed from a transparent material. In other implementations, portion 23 of substrate 22 may comprise at least one window or opening through which the detectable labels physically coupled to the functionalized structures or the bound analyte(s) may be optically detected.

In some implementations, the material or materials forming substrate 22 may be optically opaque, wherein the detectable labels chemically linked to the target analyte are detected following washing of the targeted analyte from the functionalized structures. For example, in one implementation, the analyte or analytes that have been bound to the functionalized structures of set 30-2 may be first washed with a first wash solution and then analyzed, wherein the analyte or analytes that have been bound to the functionalized structures of set 30-1 may be subsequently washed with a second wash solution and then analyzed. Such analysis may involve the detection of detection labels that have been chemically linked to the analyte either before the solution was passed along channel 24, after the analyte has bound to the functionalized structures, during the washing of the analyte from the functionalized structures, or after the analyte has been washed from the functionalized structures.

Microfluidic channel 24 is formed or extends within substrate 22. Although illustrated as being linear, microfluidic channel 24 may be curved or branched or have a serpentine path. Microfluidic channel 24 contains sets 30 and directs fluid, the solution containing the at least one analyte, through, around and across sets 30 of functionalized structures.

Microfluidic channel 24 may be formed by performing etching, microfabrication processes (e.g., photolithography), or micromachining processes in substrate 22. Accordingly, channel 22 may be defined by surfaces fabricated in the substrate of a microfluidic device. In some implementations, microfluidic channel 22 may be formed by an overall package, wherein multiple connected package components combine to form or define the microfluidic channel.

In some examples described herein, at least one dimension of a microfluidic channel and/or capillary chamber may be of sufficiently small size (e.g., of nanometer sized scale, micrometer sized scale, millimeter sized scale, etc.) to facilitate pumping of small volumes of fluid (e.g., picoliter scale, nanoliter scale, microliter scale, milliliter scale, etc.). For example, some microfluidic channels may facilitate capillary pumping due to capillary force. In addition, examples may couple at least two microfluidic channels to a microfluidic output channel via a fluid junction.

Sets 30 comprise different groupings of individual functionalized structures. Each of the functionalized structures is a structure that has been treated with a binding agent that binds to a specific molecule in a solution that may contain a complex mixture of molecules. In some implementations, the binding agent may be an antibody that binds to an epitope of an antigen analyte. In other implementations, the binding agent may be an antigen that binds to an antibody analyte. In some implementations, the binding agent, whether an antibody or an antigen, is chemically linkable to a detectable label. Such labels may emit radiation, produce a color change in a solution, fluoresce under light or, when induced, emit light. Such labels facilitate the detection of a bound analyte to measure the presence or concentration of the analyte in a solution.

The individual functionalized structures of sets 30 may have a variety of forms. In one implementation, the functionalized structures may comprise magnetic beads. In another implementation, the functionalized structures may comprise non-magnetic beads. In such implementations, each individual structure/bead of the sets of functionalized structures has a diameter of less than or equal to 10 μm. In yet another implementation, the functionalized structures may comprise posts or pillars. In some implementations, the functionalized structures may comprise a mixture of at least one of magnetic beads, non-magnetic beads and pillars. In one implementation, each individual set 30 is homogenous, wherein each of the individual functionalized structures has the same size and shape and wherein the arrangement and density of functionalized structures is uniform across the set 30. In one implementation, sets 30-1 and 30-2 are different with respect to one another in at least one characteristic other than relative location. For example, in one implementation, the individual functionalized structures of set 30-1 may have a different size and/or shape as compared to those functionalized structures of set 30-2. In one implementation, the functionalized structures of set 30-1 may have a different number, arrangement or layout, and/or density as compared to the functionalized structures of set 30-2. In one implementation, the functionalized structures of set 30-1 may have a different size and/or shape as well as at least one of a different number, arrangement or density as compared to the functionalized structures of set 30-2. In one implementation, sets 30 may have similar individual functionalized structures, but wherein sets 30 have different densities and/or layout of the individual functionalized structures.

In yet other implementations, each of sets 30-1 and 30-2 may be heterogeneous in that each of sets 30 has a mixture or combination of different sized and shaped functionalized structures, wherein the mixture of functionalized structures of set 30-1 is different than the mixture of functionalized structures of set 30-2. For example, in one implementation, set 30-1 may have types A and B of functionalized structures while set 30-2 has types C and D of functionalized structures, wherein each of types A, B, C and D of functionalized structures are different from one another with respect to at least one of the size, shape, density, number, layout, mixture or combination ratios and binding agents. In one implementation, set 30-1 may have type A and B of functionalized structures will set 30-2 has types B and C of functionalized structures. In one implementation, sets 30 may have similar combinations of different types of functionalized structures, but wherein sets 30 have different relative numbers of the different types of functionalized structures. For example, set 30-1 may have X % of type A functionalized structure and Y % of a type B functionalized structure while set 30-2 has R % of the type A functionalized structure and T % of the type B functionalized structure, wherein the variables X and Y are different than the variables R and T, respectively.

In one implementation, the functionalized structures of sets 30 may be functionalized, may be provided with binding agents, or combinations of binding agents, that are similar to one another. In another implementation, the functionalized structure sets 30 may be functionalized with different binding agents or with different combinations of different binding agents that bind to different predefined or preselected analytes.

By varying at least one of the size, shape, density, layout, mixture or combination ratios and binding agents amongst the sets 30, platform 20 may be customized so as to focus on a target analyte or a group of target analytes within or across a single platform. Although platform 20 is illustrated as having two sets 30 in series along channel 24, in other implementations, platform 20 may comprise a greater number of sets 30 in series along channel 24, wherein each of the sets 30 is different from the others. In some implementations, platform 20 may comprise sets of functionalized structures along channel 24 that are similar to one another, but that are spaced from one another along channel 24. In one implementation, platform 20 may comprise two similar sets of functionalized structures spaced by an intervening set of functionalized structures that is different than the two similar sets of functionalized structures, in at least one of individual functionalized structure shape/size and/or in at least one of functionalization (selected binding agents), number, layout and/or density of functionalized structures.

Fluid actuator 40 comprises at least one fluid actuator that moves a solution containing (or possibly containing) at least one target analyte through and across sets 30 of functionalized structures. Fluid actuator 40 is directly formed upon substrate 22 and is electrically driven. Fluid actuator 40 may incorporate electrical switches or transistors, formed in, on, or mounted to substrate 22, which control the actuation of fluid actuator 40.

The fluid actuator 40 on the platform 20 used to displace fluid through and across the sets 30 of functionalized structures, also on the platform 20, may comprise a thermal resistive fluid actuator, a piezo-membrane based actuator, and electrostatic membrane actuator, mechanical/impact driven membrane actuator, a magnetostrictive drive actuator, and electrochemical actuator, and external laser actuators (that form a bubble through boiling with a laser beam), other such microdevices, or any combination thereof.

In the example illustrated, fluid actuator 40 is illustrated as an inertial pump formed up on the platform that pumps fluid through and across the sets 30 of functionalized structures. In one implementation, the inertial pump may push fluid through and across the functionalized structures. In another implementation, as shown by broken lines, platform 20 may additionally or alternatively include fluid actuator 40′ which forms an inertial pump or ejection pump that draws fluid through and across the functionalized structures.

In some examples, the fluid actuator(s) 40, 40′ forming an inertial pump includes a thermal actuator having a heating element (e.g., a thermal resistor) that may be heated to cause a bubble to form in a fluid proximate the heating element. In such examples, a surface of a heating element (having a surface area) may be proximate to a surface of a fluid channel in which the heating element is disposed such that fluid in the fluid channel may thermally interact with the heating element. In some examples, the heating element may comprise a thermal resistor with at least one passivation layer disposed on a heating surface such that fluid to be heated may contact a topmost surface of the at least one passivation layer. Formation and subsequent collapse of such bubble may generate flow of the fluid. As will be appreciated, asymmetries of the expansion-collapse cycle for a bubble may generate such flow for fluid pumping, where such pumping may be referred to as “inertial pumping.”

In other examples, the fluid actuator(s) 40, 40′ forming an inertial pump and an ejection pump, respectively, may comprise piezo-membrane based actuators, electrostatic membrane actuators, mechanical/impact driven membrane actuators, magnetostrictive drive actuators, electrochemical actuators, external laser actuators (that form a bubble through boiling with a laser beam), other such microdevices, or any combination thereof. In some implementations, the fluid actuators 40, 40′ may displace fluid through movement of a membrane (such as a piezo-electric membrane) that generates compressive and tensile fluid displacements to thereby cause inertial fluid flow.

As will be appreciated, the fluid actuators 40, 40′ forming the inertial pump and the ejection pump, respectively, may be connected to a controller, and electrical actuation of the fluid actuator by the controller may thereby control pumping of fluid. Actuation of the fluid actuator may be of relatively short duration. In some examples, the fluid actuator may be pulsed at a particular frequency for a particular duration. In some examples, actuation of the fluid actuator may be 1 microsecond (μs) or less. In some examples, actuation of the fluid actuator may be within a range of approximately 0.1 microsecond (μs) to approximately 10 milliseconds (ms). In some examples described herein, actuation of the fluid actuator includes electrical actuation. In such examples, a controller may be electrically connected to a fluid actuator such that an electrical signal may be transmitted by the controller to the fluid actuator to thereby actuate the fluid actuator. Each fluid actuator of an example microfluidic device may be actuated according to actuation characteristics. Examples of actuation characteristics include, for example, frequency of actuation, duration of actuation, number of pulses per actuation, intensity or amplitude of actuation, phase offset of actuation.

In other implementations, the electrically driven fluid actuator 40′ may be part of a fluid ejector 42′ that ejects droplets of fluid, creating a low pressure or negative pressure that draws fluid through and across sets 30 of the functionalized structures. For example, in one implementation, fluid ejector 42′ may comprise fluid actuator 40′ in the form of a thermal resistor that vaporizes the adjacent fluid to create a bubble that displaces adjacent liquid to eject at least one drop of the liquid through an adjacent orifice 44′, creating a low pressure or sub-atmospheric pressure that draws/pulls fluid through and across the sets 30 of the functionalized structures.

FIG. 2 is a flow diagram of an example immunoassay method 100 that facilitates efficient and economical immunoassays. Method 100 may provide high throughput with the ability to multiplex a greater number of analytes in a single test. Method 100 may provide such immunoassay multiplexing with a proportional lower degree of complexity and cost.

In contrast to immunoassays that are carried out utilizing magnetic functionalized beads, method 100 may offer greater flexibility in that method 100 may be utilized with a larger variety of functionalized structures, such as non-magnetic beads and pillars. In contrast to immunoassays that are carried out with magnetic functionalized beads in a container or well plate, method 100 may be carried out on a platform having an electrically driven fluid actuator which controllably moves the solution containing the at least one analyte through and across the functionalized structures. In contrast to other immunoassays, method 100 may omit the step of sorting different assay beads after analyte binding when multiplexing different analytes. Although method 100 is described in the context of being carried out with platform 20, it should be appreciated that method 100 may likewise be carried out with any of the following described microfluidic immunoassay platforms or with similar microfluidic immunoassay platforms.

As indicated by block 104, first and second sets 30 of functionalized structures are provided along a channel 24 of a substrate, such as substrate 22. In one implementation, sets 30 are in series along channel 24.

As indicated by block 108, a fluid or solution containing an analyte (or potentially containing an analyte) is moved along the channel 24 with an electrically driven fluid actuator 40 and/or 40′ contained on the substrate 22. The rate at which the solution is to move the through and across the first and second sets of functionalized structures may be controlled to enhance binding of the at least one target analyte (if present) to the functionalized structures.

Thereafter, the fluid discharged from channel 24 may be analyzed to identify the at least one analyte that may have been bound within channel 24 to the functionalized structures sets 30. In some implementations, the analyte bound to the sets 30 of functionalized structures may be washed and removed from channel 24, wherein the wash fluid or solution containing the previously bound analyte or analytes may be analyzed. In one implementation, the wash fluid or solution is selective, removing and carrying away either the analyte bound to the functionalized structures of set 30-1 or the analyte bound to the functionalized structures of set 30-2. For example, in one implementation, the analyte or analytes that have been bound to the functionalized structures of set 30-2 may be first washed with a first washed solution and then analyzed, wherein the analyte or analytes that have been bound to the functionalized structures of set 30-1 may be subsequently washed with a second wash solution and then analyzed. Such analysis may involve the detection of detection labels that have been chemically linked to the analyte either before the solution was passed along channel 24, after the analyte has bound to the functionalized structures, during the washing of the analyte from the functionalized structures, or after the analyte has been washed from the functionalized structures. In some implementations, the at least one analyte may be detected while in a bound state to either of the sets 30.

FIGS. 3A and 3B schematically illustrate portions of an example microfluidic immunoassay platform 220. FIG. 3A is a top sectional view while FIG. 3B is a side sectional view of platform 220. Platform 220 is similar to platform 20 described above except that platform 220 is specifically illustrated as comprising sets 230-1 and 230-2 of functionalized structures 250-1 and 250-2 (collectively referred to as sets 230 and structures 250), respectively. Those remaining components of platform 220 which correspond to components of platform 20 are numbered similarly.

Functionalized structures 250 comprise columns, posts, or pillars. Functionalized structures 250-1 have diameters that are larger than the diameters of functionalized structures 250-2. Set 230-1 has a first number of structures 250-1 while set 230-2 has a second number, larger than the first number, of structures 250-2. Set 230-1 has a first density of structures 250-1 while set 230-2 has a second density of structures 250-2, larger than the first density of structures 250-1. The different sizes of structures 250 as well the different number and density of structures 250 as between sets 230-1 and 230-2 causes solution or fluid containing or potentially conveying an analyte to have different flow characteristics through and across sets 230-1 and 230-2. In other implementations, sets 230 may have similar structures 250 with similar numbers, diameters and densities. In the example illustrated, structures 250 extend a full height of channel 24, increasing likelihood of contact between the analyte in the fluid and the outer surface of the structure 250. In other implementations, structure 250 may have a height less than the height of channel 24.

As shown by FIG. 3B, structures 250-1 of set 230-1 each have an outer circumferential surface which is functionalized with a first binding agent 252-1. Structures 250-2 of set 230-2 each have an outer circumferential surface functionalized with a second binding agent 252-2, different than the first binding agent 252-1. The different binding agents 252 are chosen so as to bind to different analytes. As described above, in one implementation, the binding agents may comprise different antibodies. In another implementation, the binding agent may comprise different antigens.

In use, fluid actuator 40 moves a solution containing or potentially containing different target analytes along channel 24 through and across functionalized structures 250-1 and 250-2. Due to the different binding agents of the different sets 230, different analytes are bound to functionalized structures 250-1 as compared to functionalized structures 250-2. The different bound analytes may then be analyzed. In one implementation, the different bound analytes are subsequently washed from their respective sets 230-1, 230-2 and analyzed. In one implementation, two separate washing steps are carried out to separately remove the different analytes bound to the different sets 230. In one implementation, distinct detectable labels are chemically linked to the distinct analytes to distinguish between the analytes in a single wash solution. For example, a first detectable label that chemically links to the first analyte but not a second analyte may be used to identify the first analyte while a second detectable label that chemically links to the second analyte but not the first analyte may be used to identify the second analyte. The detectable labels facilitate detection and analysis of the presence and/or concentration of the analytes that were in the initial solution. In one implementation, the fluid actuator 40 may be additionally used to pump the different analyte washing fluids through and across structures 250 to controllably remove or release the bound analytes.

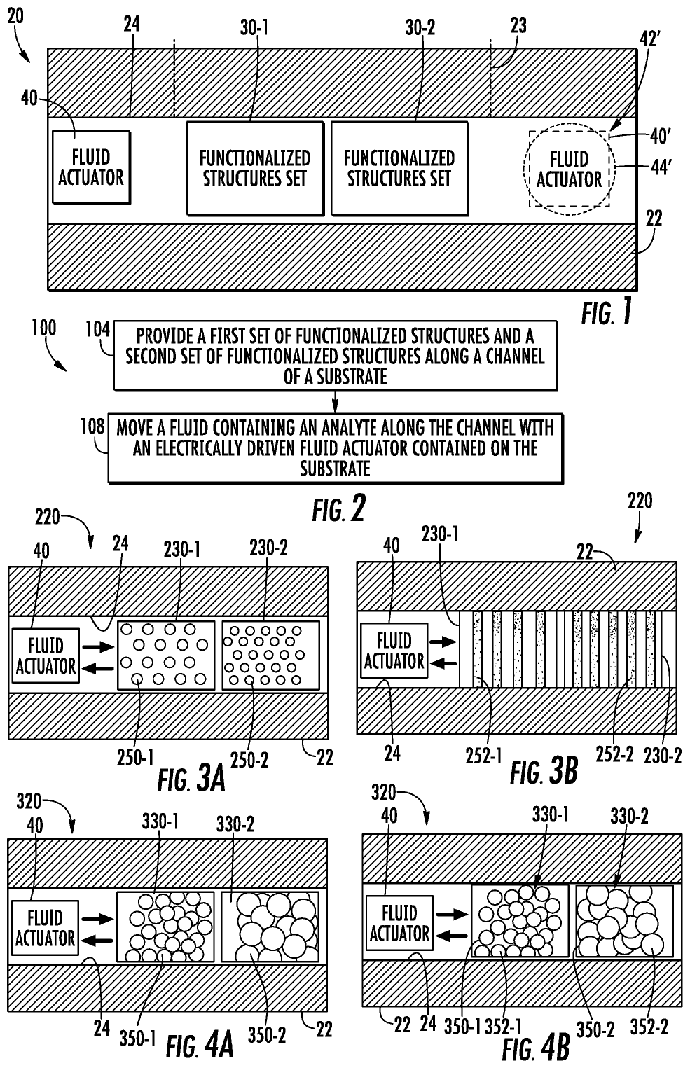

FIGS. 4A and 4B schematically illustrate portions of an example microfluidic immunoassay platform 320. FIG. 4A is a top sectional view while FIG. 4B is a side sectional view of platform 320. Platform 320 is similar to platform 20 described above except that platform 320 is specifically illustrated as comprising sets 330-1 and 330-2 of functionalized structures 350-1 and 350-2 (collectively referred to as sets 330 and structures 350), respectively. Those remaining components of platform 320 which correspond to components of platform 20 are numbered similarly.

Functionalized structures 350 comprise spheres or beads. In one implementation, each individual structure/bead of the sets of functionalized structures has a diameter of less than or equal to 10 μm. Functionalized structures 350-1 have diameters that are smaller than the diameters of functionalized structures 350-2. Set 330-1 has a first number of structures 350-1 while set 330-2 has a second number, smaller than the first number, of structures 350-2. Set 330-1 has a first density of structures 350-1 while set 330-2 has a second density of structures 350-2, less than the first density of structures 350-1. The different sizes of structures 350 as well the different number and density of structures 350 as between sets 330-1 and 330-2 causes solution or fluid containing or potentially conveying an analyte to have different flow characteristics through and across sets 330-1 and 330-2. In other implementations, sets 330 may have similar structures 350 with similar numbers, diameters and densities.

As shown by FIG. 4B, structures 350-1 of set 330-1 each have an outer surface which is functionalized with a first binding agent 352-1. Structures 350-2 of set 330-2 each have an outer surface functionalized with a second binding agent 352-2, different than the first binding agent 352-1. The different binding agents 352 are chosen so as to bind to different analytes. As described above, in one implementation, the binding agents may comprise different antibodies. In another implementation, the binding agent may comprise different antigens.

In use, fluid actuator 40 moves a solution containing or potentially containing different target analytes along channel 24 through and across functionalized structures 350-1 and 350-2. Due to the different binding agents of the different sets 330, different analytes are bound to functionalized structures 350-1 as compared to functionalized structures 350-2. The different bound analytes may then be analyzed. In one implementation, the different bound analytes are subsequently washed from their respective sets 330-1, 330-2 and analyzed. In one implementation, two separate washing steps are carried out to separately remove the different analytes bound to the different sets 330. In one implementation, distinct detectable labels are chemically linked to the distinct analytes to distinguish between the analytes in a single wash solution. For example, a first detectable label that chemical links to the first analyte but not a second analyte may be used to identify the first analyte while a second detectable label that chemical links to the second analyte but not the first analyte may be used to identify the second analyte. The detectable labels facilitate detection and analysis of the presence and/or concentration of the analytes that were in the initial solution. In one implementation, the fluid actuator 40 may be additionally used to pump the different analyte washing fluids through and across structures 350 to controllably remove or release the bound analytes.

FIG. 5 is a top sectional view illustrating portions of an example microfluidic immunoassay platform 420. Like the above described microfluidic immunoassay platforms, platform 420 facilitates efficient and economical immunoassays. Platform 420 may provide high throughput with the ability to multiplex a greater number of analytes in a single test. Platform 420 may provide such immunoassay multiplexing with a proportional lower degree of complexity and cost. Platform 420 facilitates multiple immunoassays in parallel with one another. Microfluidic immunoassay platform 420 comprises substrate 22, sample supply 423, microfluidic channels 424-1, 424-2, 424-3, 424-4 (collectively referred to as channels 424). Substrate 22 is described above. Sample supply 423 comprises a volume, passage or slot through which a sample or solution is supplied, the sample or solution potentially containing at least one analyte being targeted for identification or analysis. Sample supply 423 supplies the sample or solution to each of channels 424.

Each of channels 424 is similar to channel 24 described above. Each of channels 424 contains a series of sets of functionalized structures through which solution from supply 423 is moved by at least one fluid actuator. Channel 424-1 contains sets 430-1A, 430-1B and 430-1C (collectively referred to as sets 430-1) of functionalized structures 450-1A, 450-1B and 450-1C (collectively referred to as structures 450-1), respectively. Sets 430-1A, 430-1B and 430-1C are spaced along channel 424-1 and retained against downstream movement, in the direction indicated by arrow 425, by filters 460-1A, 460-1B and 460-1C, respectively. Filter 460-1A forms passages therethrough that are sized and spaced so as to impede the passage of functionalized structures 450-1A while allowing structures 450-1B and 450-1C to pass. Filter 460-1B forms passages therethrough that are sized and spaced so as to impede the passage of functionalized structures 450-1B while allowing structures 450-1C to pass. Filter 460-1C forms passages therethrough that are sized and spaced so as to impede the passage of functionalized structures 450-1C (as well as structures 450-1B and 450-1A) while allowing the liquid or fluid carrying the functionalized structures to pass. Filter 460-1A is located along channel 424-1 between sets 430-1A and 430-1B. Filter 460-1B is located along channel 424-1 between sets 430-1B and 430-1C. 2460-1C is located downstream of filters 460-1A in 460-1B. In one implementation, each of filters 460 may comprise pillars spaced to provide the filtering openings or passages. In another implementation, filters 460 may comprise screens or other structures which provide the noted filtering.

In the example illustrated, functionalized structures 450-1 each comprise non-magnetic beads having functionalized surfaces. In one implementation, each individual structure/bead of the sets 430 of functionalized structures has a diameter of less than or equal to 10 μm. Structures 450-1A are larger than structures 450-1B, which are larger than structures 450-1C. In one implementation, the different structures 450-1A, 450-1B and 450-1C are differently functionalized, having different analyte binding agents. In other implementations, structures 450-1A, 450-1B, and 450-1C are functionalized in a similar fashion with a similar binding agent or agents.

Channel 424-1 receives a solution containing analytes or potentially containing analytes as pumped by fluid actuator 440-1. Actuator 440-1 comprises an electrically driven fluid actuator and is similar to actuator 40 described above. In one implementation, actuator 440-1 forms an inertial pump that pumps a sample or solution along channel 424-1 through each of sets 430-1. As a solution containing or potentially carrying analytes flows through sets 430-1, analytes within the solution bind to the binding agents of the functionalized structures 450-1. In implementations where each of sets 430-1 have differently functionalized surfaces with different binding agents, different analytes are captured or retained by each of the different sets 430-1.

Channel 424-2 is connected to sample supply 423. Channel 424-2 retains sets 430-2A, 430-2B and 430-2C (collectively referred to as sets 430-2) of functionalized structures 450-2A, 450-2B and 450-2C (collectively referred to as functionalized structures 450-2), respectively. Functionalized structures 450-2 comprise non-magnetic beads having functionalized surfaces. In the example illustrated, the different sets 430-2 of functionalized structures 450-2 are differently sized with structures 450-2A being larger than structures 450-2B, which are larger than structures 450-2C. In the example illustrated, each of the different sets 430-2 of functionalized structures 450-2 are differently functionalized, having different binding agents. In other implementations, at least two, and in one implementation, all three of sets 430 have functionalized structures 450-2 which are similarly functionalized. In one implementation, sets 430-2A, 430-2B, and 430-2C are functionalized similar to sets 430-1A, 430-1B, and 430-1C, respectively, providing verification and direct comparison of the results from channels 424-1 and 424-2. In another implementation, sets 430-2 may be functionally differently than sets 430-1, facilitating the detection of the presence and/or concentration of different analytes in the solution supplied to supply 423.

Channel 424-2 includes filter 460-2C. Filter 460-2C is similar to filter 460-1C described above. Filter 460-2C has openings sized so as to impede the passage of functionalized structures 450-2C of set 430-2C. As a result, filter 460-2C blocks the passage of all functionalized structures 450-2 upstream. As shown by FIG. 5, structures 450-2C are stacked against filter 460-2C. structures 450-2B are stacked against structures 450-2C. structures 450-2A are stacked against structures 450-2B. In the example illustrated, functionalized structures 450-2 are stacked in an order from largest to smallest in the downstream direction 425. Such an order may facilitate the flow of solution along channel 424-2 to the smallest functionalized structures 450-2C. In other implementations, the size order of sets 430-2 may have other arrangements, such as smallest to largest in the downstream direction or non-ordered size progression.

Similar to channel 424-1, channel 424-2 receives solution containing analytes (or potentially containing analytes), that is pumped by fluid actuator 440-2. Fluid actuator 440-2 is similar to fluid actuator 440-1. In the example illustrated, fluid actuator 440-2 comprises an inertial pump that moves fluid through channel 424-2 in the downstream direction as indicated by arrow 425. In one implementation, fluid actuator 440-2 comprises a thermal resistor.

Channel 424-3 is similar to channel 424-1 except that fluid is moved through channel 424-3 by fluid actuator 440-3 and orifice 442-3, downstream of the sets 430-3A, 430-3B and 430-3C (collectively referred to as sets 430-3) of functionalized structures 450-3A, 450-3B and 450-3C (collectively referred to as structures 450-3), respectively. Fluid actuator 440-3 and orifice 442-3 cooperate to form a fluid ejector 444-3. Although fluid ejector 444-3 is illustrated as being provided at an end of a closed end channel 424-3, in other implementations, channel 424-3 may continue further downstream of the fluid ejector 444-3. The fluid ejector 444-3 ejects droplets of fluid through orifice 442-3 so as to draw fluid from supply 423 through and across each of the sets 430-3.

Functionalized structures 450-3 comprise non-magnetic beads having functionalized surfaces. In the example illustrated, the different sets 430-3 of functionalized structures 450-3 are differently sized with structures 450-3A being larger than structures 450-3B, which are larger than structures 450-3C. In the example illustrated, each of the different sets 430-3 of functionalized structures 450-3 are differently functionalized, having different binding agents. In other implementations, at least two, and in one implementation, all three of sets 430-3 have functionalized structures 450-3 which are similarly functionalized. In one implementation, sets 430-3A, 430-3B and 430-3C are functionalized similar to sets 430-1A, 430-1B and 430-1C, respectively, providing verification and direct comparison of the results from channels 424-1 and 424-3. In another implementation, sets 430-3 may be functionally differently than sets 430-1, facilitating the detection of the presence and/or concentration of different analytes in the solution supplied to supply 423. Although sets 430-3 are illustrated as being retained along channel 424-3 by filters 460-3A, 460-3B and 460-3C, in other implementations, sets 430-3 may be retained in a fashion similar to that shown with respect to channel 424-2, wherein filters 460-1A and 460-1B are omitted such that set 430-3B stacks against set 430-3C and set 430-3A stacks against set 430-3B.

Microfluidic channel 424-4 is similar to microfluidic channel 424-2. Channel 424-4 is similar to channel 424-1 except that fluid is additionally moved through channel 424-4 by fluid actuator 440-4B and orifice 442-4, downstream of the sets 430-4A, 430-4B, and 430-4C (collectively referred to as sets 430-4) of functionalized structures 450-4A, 450-4B, and 450-4C (collectively referred to as structures 450-4), respectively. Fluid actuator 440-4B and orifice 442-4 cooperate to form a fluid ejector 444-4. Although fluid ejector 444-4 is illustrated as being provided at an end of a closed end channel 424-4, in other implementations, channel 424-4 may continue further downstream of the fluid ejector 444-4. The fluid ejector 444-4 ejects droplets of fluid through orifice 442-4 so as to draw or pull fluid from supply 423 through and across each of the sets 430-4. In the example illustrated, movement of fluid along channel 424 is further facilitated by fluid actuator 440-4A, which is similar to fluid actuators 440-1 or 440-2. In some implementations, fluid actuator 440-4A may be omitted.

Functionalized structures 450-4 comprise non-magnetic beads having functionalized surfaces. In the example illustrated, the different sets 430-4 of functionalized structures 450-4 are differently sized with structures 450-4A being larger than structures 450-4B, which are larger than structures 450-4C. In the example illustrated, each of the different sets 430-4 of functionalized structures 450-4 are differently functionalized, having different binding agents. In other implementations, at least two, and in one implementation, all three of sets 430-4 have functionalized structures 450-4 which are similarly functionalized. In one implementation, sets 430-4A, 430-4B, and 430-4C are functionalized similar to sets 430-1A, 430-1B, and 430-1C, respectively, providing verification and direct comparison of the results from channels 424-1 and 424-4. In another implementation, sets 430-4 may be functionalized differently than sets 430-1, facilitating the detection of the presence and/or concentration of different analytes in the solution supplied by supply 423. Although sets 430-4 are illustrated as being retained along channel 424-4 by filter 460-4C, wherein upstream sets are stacked against one another as described above with respect to sets 430-2, in other implementations, sets 430-4 may be retained in a fashion similar to that shown with respect to sets 430-1 or 430-3 as described above with individual filters retaining and spacing the different sets along channel 424-4.

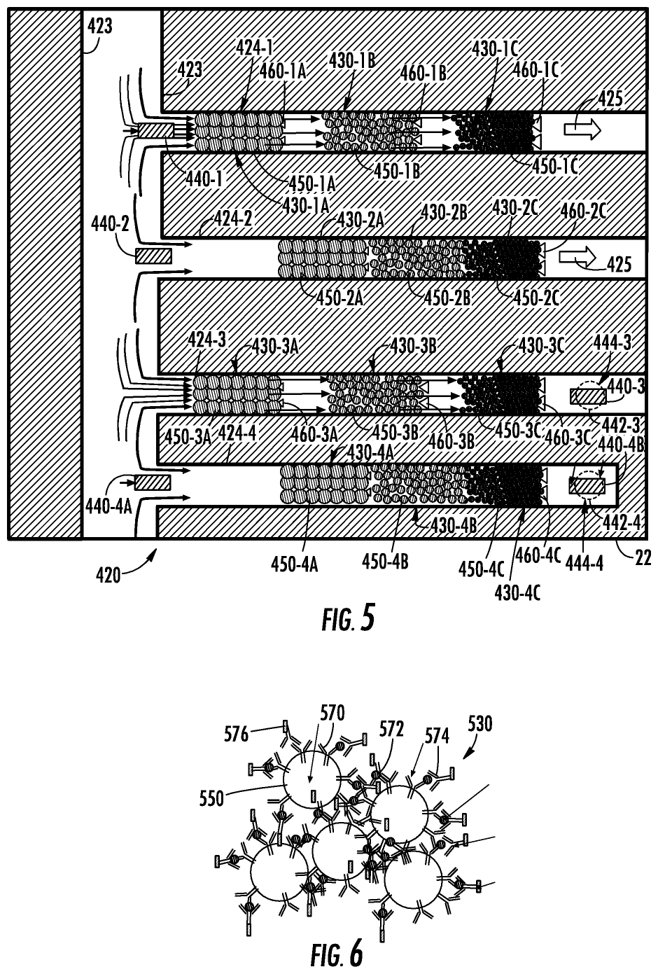

FIG. 6 illustrates portions of an example set 530 of functionalized structures 550 in the form of beads, such as non-magnetic beads. The functionalized structures 550 are each functionalized with capture elements in the form of antibodies 570. In other implementations, capture elements may be in the form of antigens. A solution containing an analyte or potentially containing an analyte, such as an antigen, is directed through and across functionalized structures 550. FIG. 6 illustrates an example where the solution contains the analyte 572, which results in the analyte 572 binding to the capture elements, antibodies 570. FIG. 6 further illustrates conjugated second antibodies 574, which are bound to the analyte/antigen 572 and to which detection labels 576, such as florescence, have been linked. Such functionalized structures 550 and the attached analyte, conjugated secondary antibodies, and detection labels, may be subsequently washed to separate the captured analyte and coupled detection labels for analysis. In some implementations, the linking of the detection labels and conjugated secondary antibodies may take place after the originally captured analyte has been washed from the functionalized structures 550.

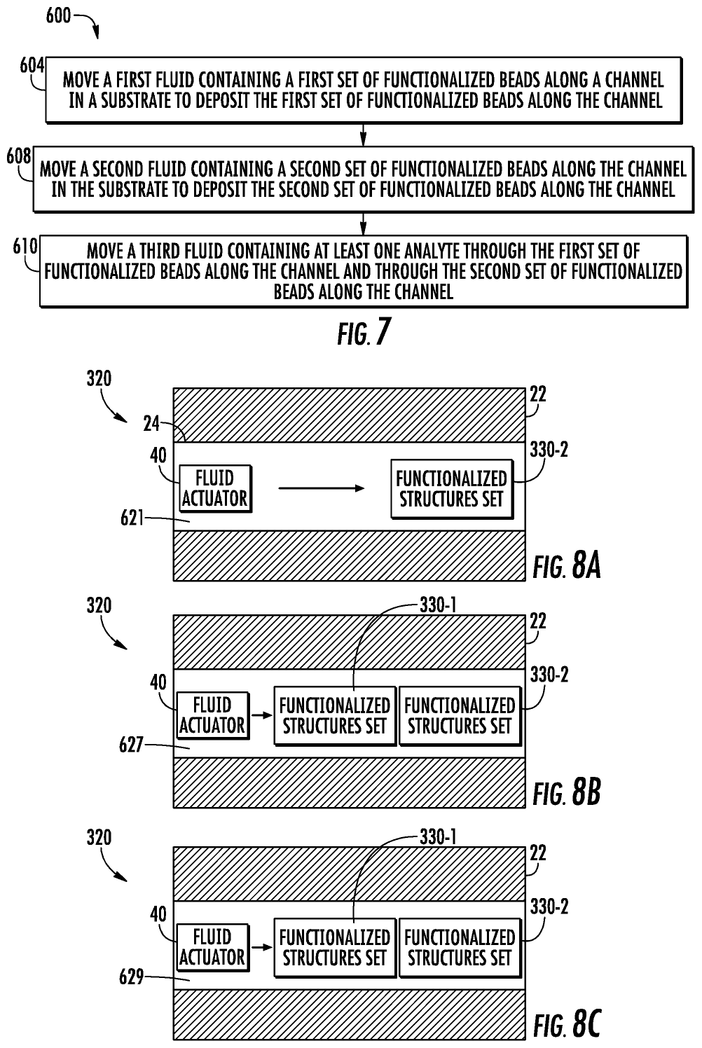

FIG. 7 is a flow diagram of an example method 600 for forming and using a microfluidic immunoassay platform, such as any of the platforms described above. FIGS. 8A, 8B and 8C are sectional views illustrating the carrying out of method 600 to form microfluidic immunoassay platform 320 described above. Method 600 facilitates efficient and economical forming of an immunoassay platform.

As indicated by block 604 and shown by FIG. 8A, a first fluid 621 containing a first set 330-2 of functionalized structures 350-2 (shown in FIGS. 4A and 4B), in the form of beads, is moved along a channel 24 of a substrate 22 to deposit the first set 330-2 along the channel 24. In one implementation, the first fluid 621 may move through channel 24 by fluid actuator 40. In one implementation, the set 330-2 may be stopped or retained by a filter, constriction, magnetization or other retention mechanism.

As indicated by block 608 and shown by FIG. 8B, a second fluid 627 containing a second set 330-1 of functionalized structures 350-1 (shown in FIGS. 4A and 4B), in the form of beads, is moved along the channel 24 of substrate 22 to deposit the first set 330-1 upstream of set 330-2 along the channel 24. In one implementation, the first fluid 621 may move through channel 24 by fluid actuator 40. In one implementation, the second set 330-1 may be retained by a filter, constriction, magnetization or other retention mechanism. In another implementation, the second set 330-1 may be stacked against set 330-2.

As indicated by block 610 and shown by FIG. 8C, a third fluid, such as a solution or sample fluid or liquid 629 containing at least one analyte (or potentially containing an analyte for which the present fluid is being tested) is moved along channel 24 of substrate 22 through and across both of sets 330-1 and 330-2 of functionalized structures. The analyte is bound to the functionalized surfaces of the functionalized structures. For example, antibodies and antigens may bind to one another. In implementations where the sets 330-1 and 330-2 contain differently functionalized structures, different analytes my bind to the different functionalized structures of the different sets 330. As described above, the bound analyte may then be detected to determine its presence and/or concentration either while within channel 24, after the beads of sets 330 have been removed from channel 24, or after the analytes have been washed from the beads of sets 330.

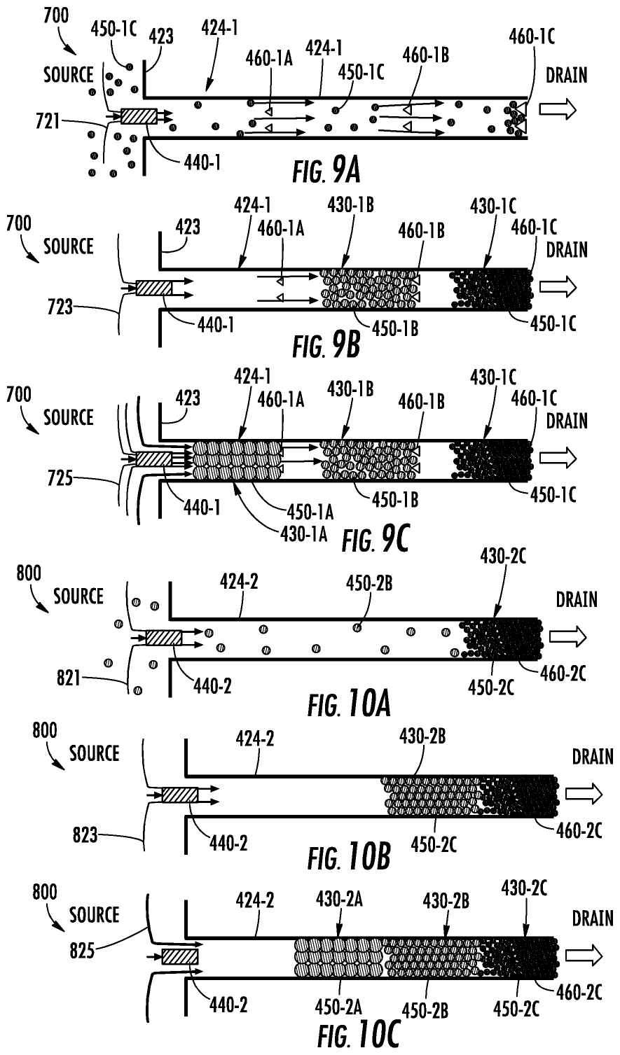

FIGS. 9A, 9B and 9C are top sectional views illustrating one example method 700 for forming at least a portion of a microfluidic immunoassay, such as channel 424-1 and its associated functionalized structures 450-1 as described above with respect to platform 420 in FIG. 5. As shown by FIG. 9A, a source, supply 423, is supplied with a first fluid 721, in the form of a liquid, containing or suspending functionalized structures 450-1C. Fluid actuator 440-1 is actuated to pump functionalized structures 450-1C along channel 424-1 through filter 460-1A and through filter 460-1B. Due to their size relative to filter 460-1C or the passages therethrough, filter 460-1C blocks or impedes further downstream movement of functionalized structures 450-1C, which results in structures 450-1C collecting and grouping to form set 430-1C (shown in FIG. 9B).

As shown by FIG. 9B, supply 423 is supplied with a second fluid 723, in the form of a liquid, containing or suspending functionalized structures 450-1B. Fluid actuator 440-1 is actuated to pump functionalized structures 450-1B along channel 424-1 through filter 460-1A. Due to their size relative to filter 460-1B or the passages therethrough, filter 460-1B blocks or impedes further downstream movement of functionalized structures 450-1B which results in structures 450-1B collecting and grouping to form set 430-1B (shown in FIG. 9C).

As shown by FIG. 9C, supply 423 is supplied with a third fluid 725, in the form of a liquid, containing or suspending functionalized structures 450-1A. Fluid actuator 440-1 is actuated to pump functionalized structures 450-1A along channel 424-1. Due to their size relative to filter 460-1A or the passages therethrough, filter 460-1A blocks or impedes further downstream movement of functionalized structures 450-1A which results in structures 450-1A collecting and grouping to form set 430-1A. Although each of sets 430-1 are illustrated as being formed through the supply of different volumes of fluid, 721, 723, 725 sequentially through channel 424-1, in other implementations, at least two, and in one implementation all three of the sets 430-1 may be concurrently formed, wherein the solution supplied by supply 423 contains two or more of different functionalized structures 450-1A, 450-1B and 450-1C, wherein the filters 460-1 separate and filter the functionalized structure to the different regions and different sets 430-1 along channel 424.

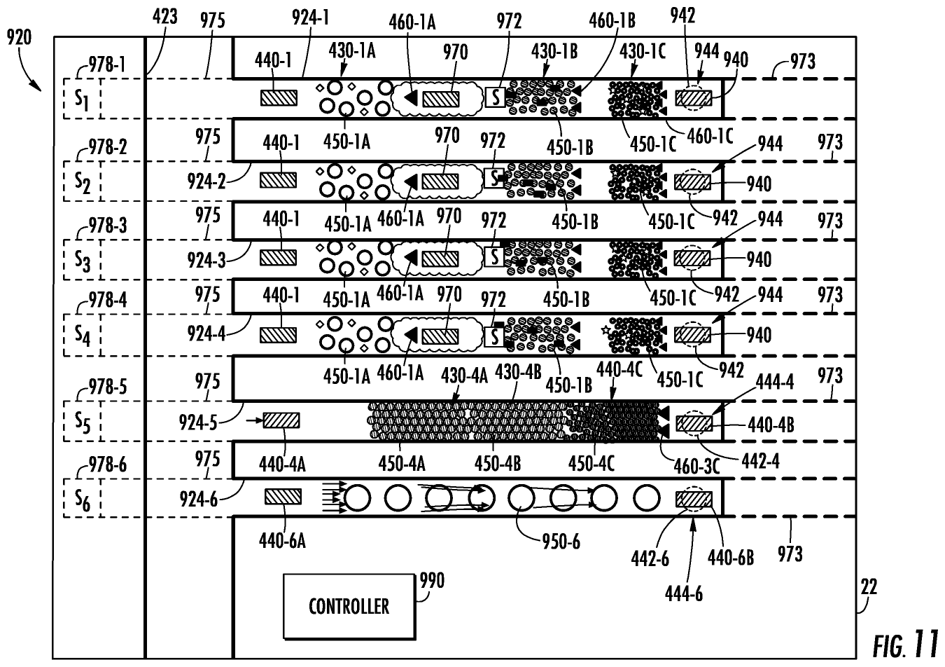

FIGS. 10A, 10B and 10C are top sectional views illustrating one example method 800 for forming at least a portion of a microfluidic immunoassay, such as channel 424-2 and its associated functionalized structures 450-2 as described above with respect to platform 420 in FIG. 5. As shown by FIG. 10A, a source, supply 423, is supplied with a first fluid 821, in the form of a liquid, containing or suspending functionalized structures 450-2C. Fluid actuator 440-2 is actuated to pump functionalized structures 450-2C along channel 424-2. Due to their size relative to filter 460-2C or the passages therethrough, filter 460-2C blocks or impedes further downstream movement of functionalized structures 450-2C which results in structures 450-2C collecting and grouping to form set 430-2C.

As shown by FIG. 10B, supply 423 is supplied with a second fluid 823, in the form of a liquid, containing or suspending functionalized structures 450-2B. Fluid actuator 440-2 is actuated to pump functionalized structures 450-2B along channel 424-2 so as to stack against structures 450-2C of set 430-2C, which results in structures 450-2B collecting and grouping to form set 430-2B.

As shown by FIG. 10C, supply 423 is supplied with a third fluid 825, in the form of a liquid, containing or suspending functionalized structures 450-2A. Fluid actuator 440-2 is actuated to pump functionalized structures 450-2A along channel 424-1 so as to stack against structures 450-2B of set 430-2B, which results in structures 450-2A collecting and grouping to form set 430-2A. although each of method 700, 800 are illustrated utilizing fluid actuators 440-1 and 440-2, respectively, to move the functionalized structures along channels 424-1 and 424-2, respectively, in other implementations, fluid within such microfluidic channels may be moved by fluid ejectors, similar to fluid ejectors 444-3 or a combination of fluid actuators similar to fluid actuator 440-1, 440-2 and fluid ejectors similar to fluid ejector 444-3.

FIG. 11 is a top sectional view illustrating portions of an example microfluidic immunoassay platform 920. Similar to platform 420 described above, platform 920 comprises a multitude of microfluidic channels 924-1, 924-2, 924-3, 924-4, 924-5 and 924-6 (collectively referred to as channels 924) which receive, in parallel, a sample fluid or solution through sample supply 423. In addition to supporting channels 924, substrate 22 further supports a controller 990. Platform may carry out method 100 described above.

Channel 924-1 is similar to channel 424-1 described above except that channel 924-1 additionally comprises fluid actuator 940, orifice 942, fluid actuator 970 and sensor 972. Those remaining components of channel 924-1 which correspond to components of channel 424-1 are numbered similarly. Fluid actuator 940 and orifice 942 are located downstream of filter 460-1C and cooperate to form a fluid ejector 944 which functions similarly to fluid ejector 444-3 described above. Fluid ejector 944 ejects droplets of fluid to draw or pull a solution containing analytes from supply 423 through and across sets 430-1. In the example illustrated, the fluid ejector 944 is located at a blind and a closed end of channel 924-1. As indicated by broken lines 973, in other implementations, channel 924-1 may continue downstream of fluid ejector 944. In the example illustrated, fluid ejector 944 and fluid actuator 440-1 may be alternately used or concurrently used to move fluid through channel 924-1. As described above, fluid ejector 944 and fluid actuator 440-1 may be additionally used when populating channel 924-1 with functionalized structures 450-1 as described above with respect to method 700 and 800.

Fluid actuator 970 comprise an electrically driven fluid actuator located downstream of filter 460-1A, between filter 460-1A and filter 460-1B, between filter 460-1A and the set 430-1B of functionalized structures 450-1B. Fluid actuator 970, upon being actuated or electrically driven, moves fluid in an upstream direction, through filter 460-1A and through set 430-1A of functionalized structures 450-1A, towards supply 423. Fluid actuator 970, when actuated, assists in dislodging the beads forming functionalized structures 450-1A to assist in cleaning and unclogging debris from filter 460-1A and from amongst functionalized structures 450-1A. in some implementations, a fluid actuator similar to fluid actuator 970 may be additionally provided downstream of filter 460-1B, between filter 460-1B and set 430-1C of functionalized structures 450-1C.

In one implementation, fluid actuator 970 is located so as to form an inertial pump that, when actuated, pumps fluid in a direction upstream, towards supply 423. In one implementation, fluid actuator 970 comprises a thermoresistive fluid actuator. In another implementation, fluid actuator 970 may comprise other types of fluid actuators as described above.

Sensor 972 comprises a device that senses the flow of fluid. In one implementation, sensor 972 comprises an impedance sensor. In another implementation, sensor 972 comprises other types of a flow sensor. Signals from sensor 972 are communicated to controller 990.

Controller 990 receive signals from sensor 972 and based upon such signals, outputs control signals controlling the actuation of fluid actuator 970. In one implementation, controller 990 comprises a non-transitory computer-readable medium that provides instructions for directing a processing unit or logic elements to control the actuation of fluid actuator 970. In one implementation, controller 990 compares the sensed flow of fluid as indicated by sensor 972 against a predetermined threshold and actuates fluid actuator 970 to reverse flow fluid within channel 924-1 upon satisfaction of the predetermined threshold. In one implementation, the magnitude of the flow detected output by sensor 972 is utilized by controller 990 as a basis for controlling the frequency, duration, or force of reverse fluid actuation by fluid actuator 970. Such reverse flow may occur while fluid actuators 940 and 440-1 are inactive.

Fluid channels 924-2, 924-3, and 924-4 are each similar to fluid channel 924-1. Each of fluid channels 924 comprises similar sets 430-1A, 430-1B, and 430-1C of functionalized structures 450-1A, 450-1B, and 450-1C, respectively. Each of fluid channels 924 comprises a reverse flow fluid actuator 970 and a sensor 972, wherein controller 990 may control the actuation of fluid actuator 970 based upon signals from sensor 972. Because a similar immunoassay or test is carried out across each of channels 424, the verification or confirmation of results across multiple channels is achieved.

As shown by broken lines 975, in lieu of each of channels 924 being supplied with a sample solution or fluid from a same reservoir supply 423, each of channels 924 may alternatively be supplied with distinct solutions or fluids from distinct fluid sources 978-1 (S1), 978-2 (S2), 978-3 (S3), 978-4 (S4), 978-5 (S5) and 978-6 (S6) (collectively referred to as sources 978). Each of such sources 978 may supply a different solution or sample. In one implementation, sources 978 may provide the same samples, but wherein the samples have been diluted to different extents. In another implementation, each of sources 978 may provide a same sample, but wherein each sample has been provided with a different reagent, a different group of reagents or different concentrations of a reagent. In such implementations, the multiple similar channels 924-1, 924-2, 924-3, and 924-4, along with the different sources 978, may provide test concordance.

Microfluidic channel 924-5 is similar to microfluidic channel 424-4 described above. In the example illustrated, channel 924-5 receives fluid from source 423, the source that also supplies fluid to each of channels 924-1, 924-2, 924-3, and 924-4. In other implementations, channel 924-5 may alternatively receive a sample or solution for testing from a separate or distinct fluid source or supply 978-5.

Microfluidic channel 924-6 receives a sample or supply of a solution from supply 423. As indicated by broken lines, in other implementations, channel 924-6 may have a dedicated fluid source or fluid supply 978-6. Microfluidic channel 924-6 contains a series or chain of functionalized structures 950-6 in the form of pillars having functionalized surfaces. Each of pillars forming functionalized structures 950-6 is similar to functionalized structures 250-1 described above. In the example illustrated, the pillars forming functionalized structures 950-6 comprise a single row of such structures. In other implementations, functionalized structures 950-6 may comprise a grid or array of such structures extending along channel 924-6. Each of such functionalized structures 950-6 is functionalized in a similar fashion, having a similar binding agent or group of binding agents.

As further shown by FIG. 11, channel 924-6 additionally comprises fluid actuator 440-6A and fluid actuator 440-6B, between which structures 950-6 are sandwiched. Fluid actuator 440-6A extends upstream of structure 950-6 and forms an inertial pump for pushing or pumping fluid through and across structures 950-6. Fluid actuator 440-6B extend proximate to orifice 442-6 and cooperates with orifice 442-6 to form a fluid ejector 444-6. Ejector 444-6, in response to control signals from controller 990, ejects droplets of fluid so as to pull or draw fluid from supply 423 (or supply 978-6) through and across the chain of functionalized structures 950-6.

Channel 924-6 and the series or chain of functionalized structures 950-6 facilitate a determination regarding a concentration of an analyte within a sample solution. In one implementation, the different extents to which an analyte has bound to the different functionalized structures 950-6 along the length of channel 924-6 is determined and, based upon this determination, the concentration of an analyte in the overall solution may be determined. A solution with a greater concentration of an analyte will result in a higher concentration of the analyte binding to the downstream functionalized structures 950-6, whereas a solution with a lesser concentration of an analyte will result in a lower concentration of the analyte binding to the corresponding downstream functionalize structures 950-6.

Although platform 920 is illustrated as comprising six microfluidic channels 924 having sets of functionalized structures, in other implementations, platform 920 may comprise a greater or fewer of such channels 924. Additional or fewer channels similar to channel 924-1 may be provided. Likewise, additional channels similar to channels 924-5 and/or channel 924-6 may be provided. Likewise, although platform 420 is illustrated as comprising four microfluidic channels 424, in other implementations, platform 420 may comprise a greater or fewer of such microfluidic channels 424. Each of such channels may have any of the architectures shown.

Although the present disclosure has been described with reference to example implementations, workers skilled in the art will recognize that changes may be made in form and detail without departing from the spirit and scope of the claimed subject matter. For example, although different example implementations may have been described as including features providing one or more benefits, it is contemplated that the described features may be interchanged with one another or alternatively be combined with one another in the described example implementations or in other alternative implementations. Because the technology of the present disclosure is relatively complex, not all changes in the technology are foreseeable. The present disclosure described with reference to the example implementations and set forth in the following claims is manifestly intended to be as broad as possible. For example, unless specifically otherwise noted, the claims reciting a single particular element also encompass a plurality of such particular elements. The terms “first”, “second”, “third” and so on in the claims merely distinguish different elements and, unless otherwise stated, are not to be specifically associated with a particular order or particular numbering of elements in the disclosure.

1. A microfluidic immunoassay platform comprising:
a substrate;
a microfluidic channel in the substrate;
a first set of functionalized structures along the channel;
a second set of functionalized structures along the channel; and
an electrically driven fluid actuator contained on the substrate to move fluid containing at least one analyte along the channel through the first set of functionalized structures and through the second set of functionalized structures.

2. The platform of claim 1, wherein the first set of functionalized structures comprise a first capture element and wherein the second set of functionalized structures comprise a second capture element different than the first capture element.

3. The platform of claim 2, wherein the first capture element and the second capture element comprise different antibodies.

4. The platform of claim 1, wherein the first set of functionalized structures comprise structures of a first size and wherein the second set of functionalized structures comprise structures of a second size, different than the first size.

5. The platform of claim 1, wherein the first set of functionalized structures comprise beads and wherein the second set of functionalized structures comprise pillars.

6. The platform of claim 1, wherein the first set of functionalized structures and the second set of structure functionalized structures comprise beads.

7. The platform of claim 6 further comprising a bead filter between the first set of functionalized structures and the second set of functionalized structures.

8. The platform of claim 6, wherein the first set of functionalized structures and the second set of functionalized structures are stacked against each other along the channel.

9. The platform of claim 1 further comprising a second electrically driven fluid actuator between the first set of functionalized structures and the second set of functionalized structures along the channel.

10. The platform of claim 1, wherein the electrically driven fluid actuator comprises an actuator selected from a group of actuators consisting of: an inertial pump and a fluid ejector.

11. The platform of claim 1 further comprising:
a second channel in the substrate;
a supply passage connected to the channel and the second channel;
a third set of functionalized structures along the second channel;
a fourth set of functionalized structures along the second channel; and
a second electrically driven fluid actuator contained on the substrate to move fluid containing an analyte along the second channel through the third set of functionalized structures and through the fourth set of functionalized structures.

12. The platform of claim 1, wherein each individual structure of the first set of functionalized structures has a diameter of less than or equal to 10 μm.

13. The platform of claim 1 further comprising a third set of functionalized structures along the channel, wherein individual structures of the third set of functionalized structures are different than individual structures of the first set of functionalized structures and the second set of functionalized structures with respect to at least one of functionalization, size and layout.

14. A microfluidic immunoassay method comprising:
providing a first set of functionalized structures and a second set of functionalized structures along a channel of a substrate; and
moving a fluid containing an analyte along the channel with an electrically driven fluid actuator contained on the substrate.

15. A microfluidic immunoassay method comprising:
moving a first fluid containing a first set of functionalized beads along a channel in a substrate to deposit the first set of functionalized beads along the channel;
moving a second fluid containing a second set of functionalized beads along the channel in the substrate to deposit the second set of functionalized beads along the channel; and
moving a third fluid containing at least one analyte through the first set of functionalized beads along the channel and through the second set of functionalized beads along the channel, using an electrically driven fluid actuator contained on the substrate.
